(19) United States
(12) Patent Application Publication
(10) Pub. No.: US 2026/0176925 A1
(43) Pub. Date: Jun. 25, 2026

(54) MACHINING GAS SWITCH LEAD WIRE DRILLING TOOL FOR INDIRECT LIVE-WIRE AND METHOD OF OPERATING THE SAME

(71) Applicant: KOREA ELECTRIC POWER CORPORATION, Naju-si (KR)

(72) Inventors: Ho-Jin JUNG, Changwon-si (KR); Chan-Soo KIM, Changwon-si (KR); Gi-Han KWON, Changwon-si (KR)

(21) Appl. No.: 18/854,944

(22) PCT Filed: Aug. 7, 2024
(86) PCT No.: PCT/KR2024/011646

§ 371 Date: Oct. 7, 2024

(30) Foreign Application Priority Data
Aug. 7, 2023 (KR) 10- 2023-0102610

(51) Int. Cl. E21B 17/04; A61B 17/32; E21B 7/06; E21B 17/02

(52) U.S. Cl. CPC E21B 17/04; E21B 7/062; E21B 7/064; E21B 17/02; A61B 2017/320028

(57) ABSTRACT

Since the present invention provides the processing gas switch lead wire drilling tool for an indirect live-wire and the method of operating the same, which can remove moisture penetrating into a lead wire (1) by drilling a sheath (3) of the lead wire (1) by a rotational force of an end mill (46) in a driller body (10) connected to an indirect live-wire insulating stick, it is possible to minimize the construction cost and time according to other preliminary work requirements and simplify the construction procedure and to decrease the failure occurrence probability compared to the peeling work due to the smaller exposure part of the charging unit when the sleeve cover is separated by storing wind and typhoon.

### TECHNICAL FIELD

The present invention relates to a processing gas switch lead wire drilling tool for an indirect live-wire and a method of operating the same, and more specifically, to a processing gas switch lead wire drilling tool for an indirect live-wire and a method of operating the same, which may remove moisture by drilling a processing gas switch lead wire by a drilling tool using an indirect live-wire method.

### BACKGROUND ART

In general, in severe cold weather, moisture penetrating into a processing gas switch (hereinafter referred to as a switch) freezes and expands according to a temperature to cause cracks and damage to bushing, thereby resulting in a ground fault.

That is, a moisture penetration path into a lead wire has a problem that moisture penetrates into a lead wire stranded wire air gap in case of lead wire compression defect (foreign sleeve), moisture penetrates into bushing due to a molded portion defect of the lead wire (mold cone) in case of lead wire molding defect, and the bushing is damaged due to moisture freezing and expansion inside the bushing in case of moisture penetration into the lead wire.

In addition, as shown in FIG. 1, for moisture penetration and electrical conduction paths, a path through which moisture penetrates into a lead wire stranded wire air gap and then moisture penetrates into the bushing is in the order of bond peeling, insulator, mold cone lifting, and moisture introduction, and a path through which moisture penetrates into the lead wire is in the order of moisture introduction into a lead wire, bonding defect of an internal conductor, and moisture introduction.

In this case, a switch case (flange) is electrically conducted by bushing damage and insulation breakage due to moisture freezing and expansion.

To prevent such failures, a compression state of a foreign metal sleeve of the switch and the presence or absence of a self-fusion tape are checked in a live-wire state, and switch bushing damage and insulation breakdown failure due to moisture penetration have been prevented by removing (peeling) a sheath of the lead wire of the switch to remove moisture inside the lead wire and then constructing a sleeve cover.

In addition, when the moisture is removed from the switch lead wire, the sleeve cover has been constructed by removing the sheath of the lead wire and removing moisture from the lead wire.

However, a direct live-wire work in which workers wear a protective gear (insulating rubber gloves, etc.) and work directly in the existing live-wire state has been prohibited (abolished) for safety reasons, and indirect live-wire work using 4 types of indirect live-wire insulating sticks (rotor stick, hot stick, grab stick, hand stick) and a tool (peeling machine) has been used as an alternative method.

Since the switch lead wire (mold cone HDCC) cannot be peeled (the sheath of the lead wire cannot be peeled) using the indirect live-wire insulating stick (rotor stick) and the tool (peeling machine) developed so far, a preliminary work for making a work section in a diagonal state is required.

That is, when the work section is in the diagonal state, workers may directly remove moisture by peeling the lead wire, but many methods and costs for the preliminary work that makes the work section in the diagonal state are unnecessarily incurred, and excessive construction costs are incurred to remove moisture penetrating into the lead wire.

### DISCLOSURE

### Technical Problem

The present invention is directed to providing a processing gas switch lead wire drilling tool for an indirect live-wire and a method of operating the same, which may remove moisture by drilling the sheath of a lead wire with indirect live-wire work, thereby minimizing a required construction cost and time and simplifying a construction procedure.

### Technical Solution

A processing gas switch lead wire drilling tool for an indirect live-wire, which is connected to an indirect live-wire insulating stick to hold a lead wire and drill one side of a sheath of the lead wire to remove moisture in an air gap, according to an embodiment of the present invention for achieving the above object includes a driller body having a first rotational body therein to be connected to the indirect live-wire insulating stick to rotate, a second rotational body having an end mill to be connected to the first rotational body to rotate simultaneously with the first rotational body, and a guide body connected to the driller body to hold and guide the lead wire to the end mill side.

The driller body and the guide body have the form of a hollow tube, and an outer circumferential surface of a rear of the driller body is overlaid on an inner circumferential surface of a front of the guide body, and the driller body and the guide body are fastened by a third bolt.

The outer circumferential surface of the driller body is formed with a flange part that guides a hole to match the third bolt for fastening as an end of the guide body is stopped in contact with the driller body when the guide body is fitted into the driller body.

The first rotational body is supported rotatably at an inner diameter center of the driller body via a plurality of bearings.

The first rotational body has a rear formed with a rotational shaft protruding backward from the driller body and connected to the second rotational body, and a front formed with a fastening surface part in which the front of the first rotational body protrudes forward from the driller body and is connected to the indirect live-wire insulating stick.

The bearing has a rear side edge tightly fixed by a partition wall formed in a longitudinal central portion of the driller body, and a front side edge tightly fixed by a snap ring fastened to an inner circumferential surface of the driller body.

The second rotational body has a front formed with a hollow shaft into which the rotational shaft is inserted and which is fixed by a first bolt, and a rear formed with a drill chuck into which the end mill is inserted and which is fixed by a second bolt.

The first bolt is configured in the form of a set screw fastened to the rotational shaft while passing through an outer circumferential surface of the hollow shaft.

The second bolt is configured in the form of a set screw fastened to the end mill while passing through one side of an outer circumferential surface of the drill chuck.

The guide body has a rear formed with an opening so that a circumference side of the lead wire enters and exits from the rear of the guide body to a predetermined depth.

The inside of the opening is formed with a semicircular part to be in close contact with a curvature of an outer circumference of the lead wire.

In addition, a processing gas switch lead wire drilling tool for an indirect live-wire, which is connected to an indirect live-wire insulating stick to hold a lead wire and drill one side of a sheath of the lead wire to remove moisture in an air gap, according to another embodiment of the present invention includes an end mill installed inside a driller body to be connected to the indirect live-wire insulating stick to rotate, and a guide body connected to the driller body to hold the lead wire to the end mill side and made of a material having a frictional force of a predetermined level or more to prevent the slippery of the lead wire.

Meanwhile, a method of operating a processing gas switch lead wire drilling tool for an indirect live-wire according to an embodiment of the present invention includes protecting a neutral or low-pressure line, drilling a sheath of a lead wire after connecting a drilling tool to an indirect live-wire insulating stick to hold the lead wire, installing an insulating cover to surround the drilled lead wire using the indirect live-wire insulating stick, tapping both ends of the insulating cover by the indirect live-wire insulating stick, and removing the protection of the neutral or low-pressure line.

### Advantageous Effects

Since the present invention provides the processing gas switch lead wire drilling tool for an indirect live-wire and the method of operating the same, which can remove moisture penetrating into the lead wire by drilling the sheath of the lead wire by the rotational force of the end mill in the driller body connected to the indirect live-wire insulating stick, it is possible to minimize the construction cost and time according to other preliminary work requirements and simplify the construction procedure and to decrease the failure occurrence probability compared to the peeling work due to the smaller exposure part of the charging unit when the sleeve cover is separated by storing wind and typhoon.

### DESCRIPTION OF DRAWINGS

FIG. 1 is an exemplary view of moisture penetration and electrical conduction paths of a conventional processing gas switch.

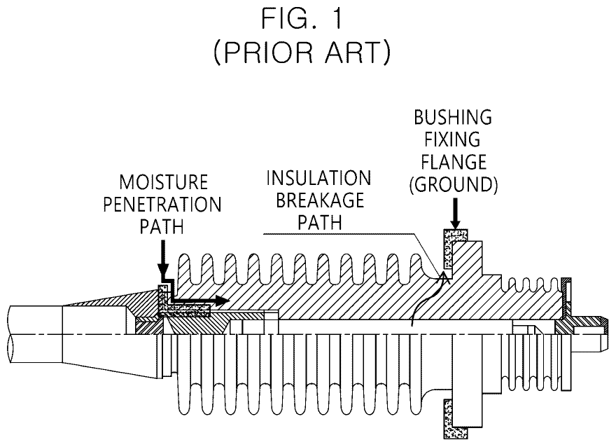

FIG. 2 is an exemplary view of coupling of a lead wire driller of a processing gas switch according to the present invention.

FIG. 3 is an exemplary view of a lead wire guide of the processing gas switch according to the present invention.

FIG. 4 is an exploded exemplary view of the lead wire driller of the processing gas switch according to the present invention.

FIG. 5 is an exemplary view of coupling between the lead wire driller and guide of the processing gas switch according to the present invention.

FIG. 6 is a cross-sectional exemplary view of coupling of the lead wire driller and guide of the processing gas switch according to the present invention.

Descriptions of reference numerals

| | |
|---|---|
| 1: lead wire | 3: sheath |
| 5: stranded wire | 7: air gap |
| 10: driller body | 12: partition wall |
| 14: flange part | 20: a first rotational body |
| 22: rotational shaft | 24: fastening surface part |
| 30: bearing | 32: snap ring |
| 40: second rotational body | 42: hollow shaft |
| 43: first bolt | 44: drill chuck |
| 45: second bolt | 46: end mill |
| 50: guide body | 52: opening |
| 54: semicircular part | 56: third bolt |

### MODE FOR INVENTION

Hereinafter, to fully understand the present invention, exemplary embodiments of the present invention will be described with reference to the accompanying drawings. Embodiments of the present invention may be modified in various forms, and the scope of the present invention should not be construed as being limited to the following embodiments described in detail. It should be noted that the same components in each drawing may be denoted by the same reference numerals. Detailed descriptions of well-known functions and configurations that are determined to unnecessarily obscure the gist of the present invention will be omitted.

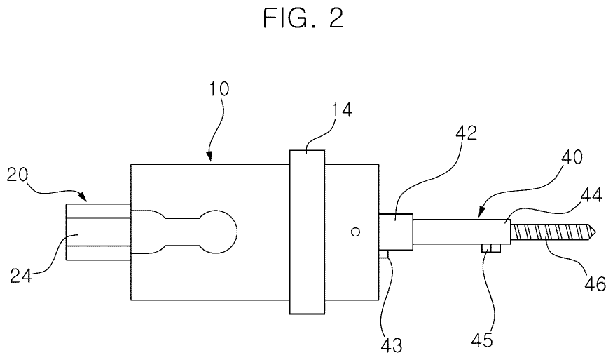

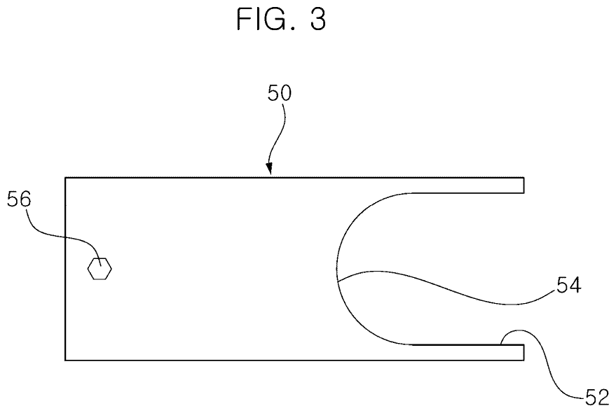

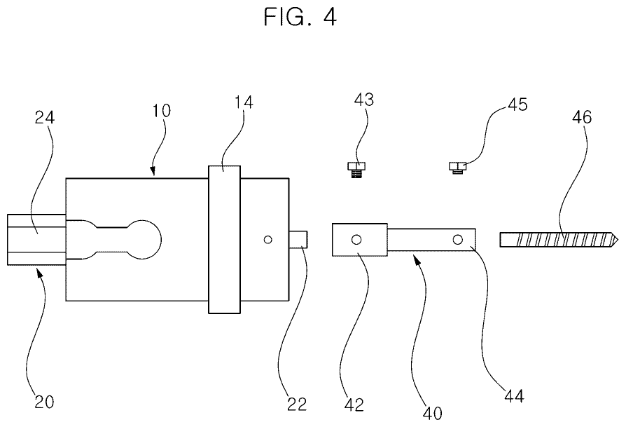

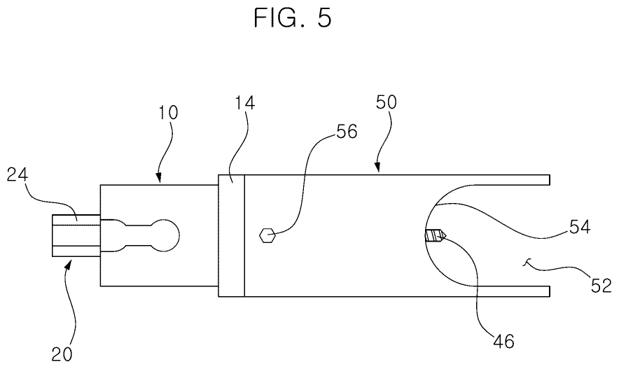

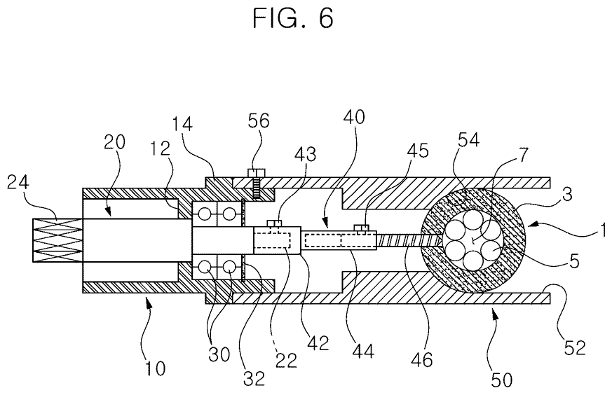

Referring to FIGS. 2 to 6, a processing gas switch lead wire drilling tool for an indirect live-wire according to the embodiment of the present invention is connected to an indirect live-wire insulating stick to hold a lead wire 1 and drill one side of a sheath 3 of the lead wire 1 to remove moisture in an air gap 7 and includes a driller body 10, a first rotational body 20, a second rotational body 40, and a guide body 50.

That is, the driller body 10 is formed in the form of a hollow tube connected to the indirect live-wire insulating stick, and a flange part 14 that guides a hole to match a third bolt 56 for fastening as an end of the guide body 50 is stopped in contact with the driller body 10 is formed on an outer circumferential surface of the hollow tube, and a ring-shaped partition wall 12 is formed on a longitudinal central portion of an inner circumferential surface of the hollow tube.

The first rotational body 20 is installed rotatably in an inner center of the driller body 10 via a plurality of bearings 30, in which a rear of the first rotational body 20 is formed with a rotational shaft 22 protruding backward from the driller body 10 and connected to the second rotational body 40, and a front thereof is formed with a hexagonal or quadrangular fastening surface part 24 in which the front of the first rotational body 20 protrudes forward from the driller body 10 and is connected to the indirect live-wire insulating stick.

The bearing 30 has a rear side edge tightly fixed by the partition wall 12 formed at the longitudinal central portion of the driller body 10 and a front side edge tightly fixed by a snap ring 32 fastened to the inner circumferential surface of the driller body 10 to allow the first rotational body 20 to smoothly rotate forward and backward in the inner center of the driller body 10.

The second rotational body 40 is connected to surround the rotational shaft 22 formed at the rear of the first rotational body 20 to rotate simultaneously with the first rotational body 20.

That is, the second rotational body 40 has a front formed with a hollow shaft 42 into which the rotational shaft 22 of the first rotational body 20 is inserted and which is fixed by the first bolt 43 and a rear formed with a drill chuck 44 into which an end mill 46 is inserted and which is fixed by a second bolt 45.

In this case, the first bolt 43 is preferably configured in the form of a set screw that is fastened to the rotational shaft 22 while passing through one side of an outer circumferential surface of the hollow shaft 42, and the second bolt 45 is preferably configured in the form of a set screw that is fastened to the rear of the end mill 46 while passing through one side of an outer circumferential surface of the drill chuck 44.

The end mill 46 may be adjusted in length by moving forward and backward through a space formed inside the drill chuck 44 according to the thickness of the sheath 3 of the lead wire 1 and detachably attached by fastening of the second bolt 45.

In addition, when the indirect live-wire insulating stick (rotor stick) connected to the fastening surface part 24 of the front of the first rotational body 20 is rotated in one direction, the end mill 46 serves to drill the sheath 3 of the lead wire 1 in contact with a sharp blade at an end of the end mill 46 by a rotational force of the second rotational body 40 that is linearly connected to the first rotational body 20 to rotate simultaneously.

The guide body 50 is connected to the driller body 10 to guide or hold the lead wire 1 toward the end mill 46 and is made of a material having a frictional force of a predetermined level or more to prevent the slippery of the lead wire 1.

That is, the guide body 50 is in the form of a hollow tube, and the inner circumferential surface of the front of the hollow tube is overlaid on the outer circumferential surface of the rear of the driller body 10, and the guide body 50 and the driller body 10 are fastened by a third bolt 56 to control the movement of the lead wire 1 during the drilling work for the lead wire 1.

The guide body 50 has a rear formed with an opening 52 so that a circumference side of the lead wire 1 may enter and exit from the rear of the guide body 50 to a predetermined depth, and the inside of the opening 52 is formed with a semicircular part 54 to be in close contact with a curvature of an outer circumference of the lead wire 1.

Meanwhile, a method of operating the processing gas switch lead wire drilling tool for an indirection live-wire according to the embodiment of the present invention includes protecting neutral or low-pressure lines, drilling the sheath 3 of the lead wire 1 after connecting the drilling tool to the indirect live-wire insulating stick to hold the lead wire 1, installing an insulating cover to surround the drilled lead wire 1 using the indirect live-wire insulating stick, taping both ends of the insulating cover using the indirect live-wire insulating stick, and removing the protection of the neutral or low-pressure line.

In this case, when the sheath 3 of the lead wire 1 is drilled, an assistant worker uses a grab stick to fix the lead wire 1, and a main worker couples the drilling tool to the rotor stick and then drills the lowest point of the sheath 3 of the lead wire 1 to easily remove moisture.

Here, during the drilling work, a blade length of the end mill 46 is preferably adjusted to 6.2 mm for 400 A and 8 mm or less for 630 A to prevent damage to a conductor wire that is a stranded wire 5 in the lead wire 1.

When the insulating cover is installed, the assistant worker uses a D-type hook to fix the indirect live-wire insulating cover, and the main worker uses a hand stick to install the indirect live-wire insulating cover.

When both ends of the insulating cover are tapped, the assistant worker uses the hand stick to hold ends of the tape, and the main worker firmly tapes the ends.

As described above, since the present invention provides the processing gas switch lead wire drilling tool for an indirect live-wire and the method of operating the same, which can remove moisture penetrating into the lead wire by drilling the sheath of the lead wire by the rotational force of the end mill in the driller body connected to the indirect live-wire insulating stick, it is possible to minimize the construction cost and time according to other preliminary work requirements and simplify the construction procedure and to decrease the failure occurrence probability compared to the peeling work due to the smaller exposure part of the charging unit when the sleeve cover is separated by storing wind and typhoon.

Meanwhile, the present invention is not limited to the above-described embodiments, but may be implemented through modifications and changes without departing from the gist of the present invention, and the technical spirit to which such modifications and changes are added should also be considered to fall within the scope of the appended claims.

1. A processing gas switch lead wire drilling tool for an indirect live-wire, which is connected to an indirect live-wire insulating stick to hold a lead wire (1) and drill one side of a sheath (3) of the lead wire (1) to remove moisture in an air gap (7), the processing gas switch lead wire drilling tool comprising:
a driller body (10) having a first rotational body (20) therein to be connected to the indirect live-wire insulating stick to rotate;
a second rotational body (40) having an end mill (46) to be connected to the first rotational body (20) to rotate simultaneously with the first rotational body (20); and
a guide body (50) connected to the driller body (10) to hold and guide the lead wire (1) to the end mill (46) side.

2. The processing gas switch lead wire drilling tool of claim 1, wherein the driller body (10) and the guide body (50) have the form of a hollow tube, and
an outer circumferential surface of a rear of the driller body (10) is overlaid on an inner circumferential surface of a front of the guide body (50), and the driller body (10) and the guide body (50) are fastened by a third bolt (56).

3. The processing gas switch lead wire drilling tool of claim 2, wherein the outer circumferential surface of the driller body (10) is formed with a flange part (14) that guides a hole to match the third bolt (56) for fastening as an end of the guide body (50) is stopped in contact with the driller body (10) when the guide body (50) is fitted into the driller body (10).

4. The processing gas switch lead wire drilling tool of claim 1, wherein the first rotational body (20) is supported rotatably at an inner diameter center of the driller body (10) via a plurality of bearings (30).

5. The processing gas switch lead wire drilling tool of claim 4, wherein the first rotational body (20) has a rear formed with a rotational shaft (22) protruding backward from the driller body (10) and connected to the second rotational body (40), and
a front formed with a fastening surface part (24) in which the front of the first rotational body (20) protrudes forward from the driller body (10) and is connected to the indirect live-wire insulating stick.

6. The processing gas switch lead wire drilling tool of claim 4, wherein the bearing (30) has a rear side edge tightly fixed by a partition wall (12) formed in a longitudinal central portion of the driller body (10), and
a front side edge tightly fixed by a snap ring (32) fastened to an inner circumferential surface of the driller body (10).

7. The processing gas switch lead wire drilling tool of claim 4, wherein the second rotational body (40) has a front formed with a hollow shaft (42) into which the rotational shaft (22) is inserted and which is fixed by a first bolt (43), and
a rear formed with a drill chuck (44) into which the end mill (46) is inserted and which is fixed by a second bolt (45).

8. The processing gas switch lead wire drilling tool of claim 7, wherein the first bolt (43) is configured in the form of a set screw fastened to the rotational shaft (22) while passing through an outer circumferential surface of the hollow shaft (42).

9. The processing gas switch lead wire drilling tool of claim 7, wherein the second bolt (45) is configured in the form of a set screw fastened to the end mill (46) while passing through one side of an outer circumferential surface of the drill chuck (44).

10. The processing gas switch lead wire drilling tool of claim 2, wherein the guide body (50) has a rear formed with an opening (52) so that a circumference side of the lead wire (1) enters and exits from the rear of the guide body (50) to a predetermined depth.

11. The processing gas switch lead wire drilling tool of claim 10, wherein the inside of the opening (52) is formed with a semicircular part (54) to be in close contact with a curvature of an outer circumference of the lead wire (1).

12. A processing gas switch lead wire drilling tool for an indirect live-wire, which is connected to an indirect live-wire insulating stick to hold a lead wire (1) and drill one side of a sheath (3) of the lead wire (1) to remove moisture in an air gap (7), the processing gas switch lead wire drilling tool comprising:
an end mill (46) installed inside a driller body (10) to be connected to the indirect live-wire insulating stick to rotate; and
a guide body (50) connected to the driller body (10) to hold the lead wire (1) to the end mill (46) side and made of a material having a frictional force of a predetermined level or more to prevent the slippery of the lead wire (1).

13. A method of operating a processing gas switch lead wire drilling tool for an indirect live-wire, the method comprising:
protecting a neutral or low-pressure line;
drilling a sheath (3) of a lead wire (1) after connecting a drilling tool to an indirect live-wire insulating stick to hold the lead wire (1);
installing an insulating cover to surround the drilled lead wire (1) using the indirect live-wire insulating stick;
tapping both ends of the insulating cover by the indirect live-wire insulating stick; and
removing the protection of the neutral or low-pressure line.
